(19) United States
(12) Patent Application Publication
(10) Pub. No.: US 2025/0161534 A1
(43) Pub. Date: May 22, 2025

(54) TISSUE ENGINEERING CARTILAGE PARTICLE GRAFT AND PREPARATION METHOD THEREFOR

(71) Applicants: SHANGHAI RESTHETIC BIO CO., LTD, Shanghai (CN); SHANGHAI NINTH PEOPLE'S HOSPITAL, SHANGHAI JIAOTONG UNIVERSITY SCHOOL OF MEDICINE, Shanghai (CN)

(72) Inventors: Guangdong ZHOU, Shanghai (CN); Yingying HUO, Shanghai (CN); Xiaodi WU, Shanghai (CN); Yu LIU, Shanghai (CN); Yao YU, Shanghai (CN)

(21) Appl. No.: 18/841,534

(22) PCT Filed: Feb. 22, 2023
(86) PCT No.: PCT/CN2023/077713

§ 371 Date: Aug. 26, 2024

(30) Foreign Application Priority Data
Feb. 25, 2022 (CN) 202210178749.X

(51) Int. Cl. A61L 27/38; A61L 27/36; C12N 5/077

(52) U.S. Cl. CPC A61L 27/3817; A61L 27/3633; A61L 27/3852; C12N 5/0655; A61L 2430/24

(57) ABSTRACT

A tissue engineering cartilage particle graft and a preparation method therefor are provide. Injectable cartilage particles are prepared from chondrocytes by means of amplification, inoculation, and in-vitro induced culture. Cartilage regeneration is achieved after the cartilage particles are implanted into a defect site, and the cartilage particles can be used for various filling treatment and cartilage repair.

### TECHNICAL FIELD

The present invention relates to the field of biomedical tissue engineering, in particular to a tissue engineering cartilage particle graft formed by self-assembly of chondrocytes, and a preparation method therefor.

### BACKGROUND

Cartilage is a tissue without blood vessels, and due to its low self-repair ability, cartilage injury or deformity has always been a major challenge in clinical treatment.

Tissue engineering technology provides a new treatment method for cartilage defect repair, among which injectable cartilage stands out due to its advantages of rapid shaping and convenient use for 3D bioprinting, etc. However, injectable cartilage can only be used for transplantation with scaffold materials as carriers, while the commonly used hydrogel scaffold is a chemically modified foreign body, which will cause problems such as inflammation, incomplete absorption of materials and long-term residual in the body.

In addition, the existing cartilage particle formed by injectable gel cartilage is actually a cartilage slice, whose size and uniformity are difficult to control, and its preparation method is mechanical cutting, which will cause a certain degree of mechanical damage to the tissue.

Therefore, there is an urgent need in this field to provide a new method for constructing a tissue engineering injectable cartilage.

### SUMMARY OF THE INVENTION

The purpose of the present invention is to provide a tissue engineering cartilage particle graft formed by self-assembly of chondrocytes.

In the first aspect of the present invention, it provides a tissue engineering cartilage particle comprising a cell population composed of chondrocytes and an extracellular matrix secreted by chondrocytes, wherein the extracellular matrix wraps around the cell population to form full and flattened spherical particles, and the average particle size of the cartilage particle is 500 μm-1 mm, wherein the density of chondrocytes in a single cartilage particle is at least 10⁴-10⁵ cells per cartilage particle.

In another preferred embodiment, the cartilage particle is prepared by forming chondrocyte micro-clusters through concentrated incubation culture of chondrocytes, and then subjecting the chondrocyte micro-clusters to in vitro chondrogenic induction culture.

In another preferred embodiment, the “concentrated incubation” refers to inoculating a chondrocyte suspension into a centrifuge tube, subsequently placing the centrifuge tube vertically and statically in an incubator, and then continuing incubation after cell precipitation.

In another preferred embodiment, during the concentrated incubation, the chondrocyte suspension is inoculated at the following concentrations: 0.05×10⁶-20×10⁶ cells/mL of culture medium, preferably 0.1×10⁶-10×10⁶ cells/mL of culture medium, and more preferably 0.2×10⁶-5×10⁶ cells/mL of culture medium.

In another preferred embodiment, during the concentrated incubation, the culture system is 10-50 ml, preferably 15-30 ml, and more preferably about 20 mL of culture medium.

In another preferred embodiment, the bottom of the centrifuge tube is conical, circular, or oval shaped.

In another preferred embodiment, the medium used for concentrated incubation is a concentrated incubation medium, which comprises the following components: DMEM high glucose medium, 1% triple antibiotic (v/v), and 10% fetal bovine serum (v/v), calculated by the total volume of the medium.

In another preferred embodiment, the in vitro chondrogenic induction culture is performed using a chondrogenic induction medium, which comprises the following components: DMEM high glucose medium, 1% triple antibiotic (v/v), 10 ng/ml TGF-β, and 50 ng/ml IGF-I, calculated by the total volume of the medium.

In another preferred embodiment, the triple antibiotic comprises penicillin, streptomycin, and amphotericin B.

In another preferred embodiment, the triple antibiotic contains 10000 units of penicillin, 10000 μg of streptomycin, and 25 μg of amphotericin B per milliliter.

In another preferred embodiment, the concentrated incubation time of chondrocytes is 3-24 hours, preferably 4-12 hours, and most preferably 6 hours.

In another preferred embodiment, the chondrogenic induction culture time is 1-4 weeks, preferably 2-3 weeks, and most preferably 2 weeks.

In another preferred embodiment, the particle size of the cartilage particle can be adjusted by controlling the time of in vitro chondrogenic induction culture.

In another preferred embodiment, the chondrocytes are derived from a human or non-human mammal.

In another preferred embodiment, the chondrocytes are derived from elastic cartilage, fibrocartilage, or hyaline cartilage.

In another preferred embodiment, the chondrocytes are selected from ear chondrocytes, costal chondrocytes, articular chondrocytes, or a combination thereof, preferably ear chondrocytes.

In another preferred embodiment, the chondrocytes are derived from the autologous or allogeneic sources.

In another preferred embodiment, the chondrocytes are obtained from the subject's autologous chondrocytes.

In another preferred embodiment, the subject suffers from a cartilage defect.

In another preferred embodiment, the cartilage defect is selected from the group consisting of: an articular cartilage defect, an auricular cartilage defect, and a nasal cartilage defect.

In the second aspect of the present invention, it provides a method for preparing the tissue engineering cartilage particle of the first aspect of the present invention, which comprises the following steps:
(1) Providing isolated chondrocytes for primary culture and subculture to obtain subcultured chondrocytes;
(2) Performing concentrated incubation culture on the subcultured chondrocytes obtained in step (1) in a concentrated incubation medium, to obtain chondrocyte micro-clusters;
(3) Culturing the chondrocyte micro-clusters for 3-5 days to obtain chondrocyte clusters;
(4) Performing in vitro chondrogenic induction culture of the chondrocyte clusters in a chondrogenic induction medium to obtain the tissue engineering cartilage particle.

In another preferred embodiment, the subcultured chondrocytes are chondrocytes from passages 1 to 3, preferably chondrocytes from passage 1.

In another preferred embodiment, in step (2), the density of the subcultured chondrocytes inoculated for the concentrated incubation culture is 0.5-1×10⁶ cells/ml.

In another preferred embodiment, the density of chondrocytes in the chondrocyte clusters is 10⁴-10⁵ cells per chondrocyte micro-cluster.

In another preferred embodiment, the concentrated incubation culture time is 3-24 hours, preferably 4-12 hours, and most preferably 6 hours.

In another preferred embodiment, the chondrogenic induction culture time is 1-4 weeks, preferably 2-3 weeks, and most preferably 2 weeks.

In another preferred embodiment, the concentrated incubation medium comprises: DMEM high glucose medium containing 1% triple antibiotic (v/v) and 10% fetal bovine serum (v/v), calculated by the total volume of the medium.

In another preferred embodiment, the chondrogenic induction medium comprises: DMEM high glucose medium containing 1% triple antibiotic (v/v), 10 ng/mL TGF-β, and 50 ng/ml IGF-I, calculated by the total volume of the medium.

In another preferred embodiment, the triple antibiotic comprises penicillin, streptomycin, and amphotericin B.

In another preferred embodiment, the triple antibiotic contains 10000 units of penicillin, 10000 μg of streptomycin, and 25 μg of amphotericin B per milliliter.

In the third aspect of the present invention, it provides a formulation or transplantation composition, which comprises:
(a) the tissue engineering cartilage particle of the first aspect of the present invention; and
(b) a pharmaceutically acceptable carrier.

In another preferred embodiment, the formulation or transplantation composition is used for repairing a cartilage defect.

In the fourth aspect of the present invention, it provides a use of the tissue engineering cartilage particle of the first aspect of the present invention in the manufacture of a medical product for repairing a cartilage defect.

In another preferred embodiment, the cartilage defect is selected from the group consisting of: an articular cartilage defect, an auricular cartilage defect, a nasal cartilage defect, and a combination thereof.

In another preferred embodiment, the medical product is a cartilage tissue graft that can be pre-molded into a shape that matches the cartilage defect site.

In another preferred embodiment, the shape of the cartilage tissue graft is consistent or substantially consistent with the shape of cartilage injury in the human auricle, nasal dorsum, nasal wing, zygomatic arch, or superciliary arch.

In another preferred embodiment, the shape of the cartilage tissue graft is selected from the group consisting of: tubular, rectangular, diamond-shaped, sheet-like, cylindrical, conical, spherical, and a combination thereof.

In the fifth aspect of the present invention, it provides a method for repairing a cartilage defect, which comprises a step of administrating the tissue engineering cartilage particle of the first aspect of the present invention or the formulation or transplantation composition of the third aspect of the present invention to a subject in need thereof.

In another preferred embodiment, the subject in need thereof suffers from a cartilage defect.

In another preferred embodiment, the cartilage defect is selected from the group consisting of: an articular cartilage defect, an auricular cartilage defect, a nasal cartilage defect, and a combination thereof.

It should be understood that within the scope of the present invention, each technical features of the present invention described above and in the following (such as examples) may be combined with each other to form a new or preferred technical solution, which is not listed here due to space limitations.

### DESCRIPTION OF THE DRAWINGS

FIG. 1 shows the gross view of cartilage particles; where A shows the culture in a 10×10 cm culture dish, with a scale bar of 1 cm in the figure, B is a partial enlargement of Figure A, with a scale bar of 100 μm, and C is the microscopic view of cartilage particles, with a scale bar of 100 μm.

FIG. 2 shows the injectability of particulate cartilage gel; where A is the cartilage particle gel collected in the syringe, and B shows the injection process into the culture dish.

FIG. 3 shows that pre-molded particulate cartilage in vitro can maintain a certain shape subcutaneously; where A represents a cartilage block with a special morphology formed by molding cartilage particles and culturing them in vitro, B represents the cartilage block that maintains its special morphology after demolding, C represents the cartilage block that can maintain its special morphology after subcutaneous culture for 4 weeks, and D represents that the cartilage has integrated into a relatively mature block-like cartilage with its morphology well-preserved. The scale bar for all above is 1 cm.

FIG. 4 shows the H-E staining results of the regenerated cartilage tissue after subcutaneous implantation of the tissue engineering cartilage particles graft in animals for 4 weeks, displaying mature and homogeneous cartilage tissue, with a scale bar of 500 μm.

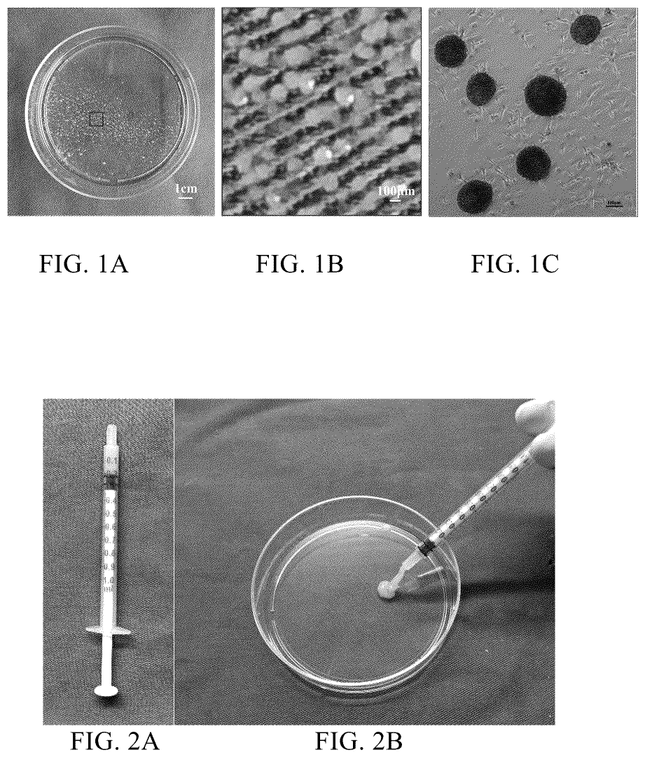

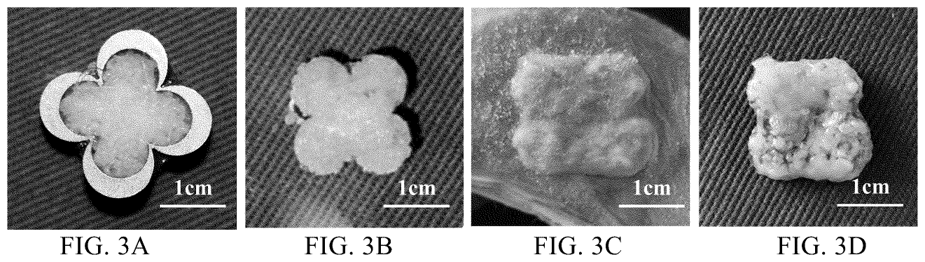

### DETAILED DESCRIPTION

Through extensive and in-depth research, the inventor unexpectedly discovered for the first time that by inoculating a specific number of chondrocytes onto and/or spreading them within a centrifuge tube with a conical (circular or oval) bottom, and after concentrated incubation, the inoculated chondrocytes adhered to each other to form cell micro-clusters. Under appropriate culture conditions, the chondrocyte micro-clusters were subjected to in vitro induced culture to form a cartilage particle rich in extracellular matrix, and the extracellular matrix is rich in glycosaminoglycans and type II collagen, as well as water, providing support and lubrication for injectable cartilage. For the cartilage particles of the present invention, by controlling the inoculation density and incubation time, the particle size thereof can be controlled to be smaller than the bore diameter of the syringe needle. Therefore, cartilage regeneration can be performed by injecting them subcutaneously through a syringe. Meanwhile, the cartilage particles of the present invention can be pre-molded in vitro to achieve regeneration of special shaped cartilage, further forming special shaped cartilage tissue in vivo. The present invention has been completed on this basis.

### Term

Unless otherwise defined, all technical and scientific terms used herein have the same meanings as those commonly understood by those skilled in the art to which the present invention belongs.

As used herein, “tissue engineering cartilage particle”, “injectable cartilage particle” and “the cartilage particle of the present invention” can be interchangeably used, all of which refer to the cartilage particle of the first aspect of the present invention.

As used herein, the term “cartilage (stem) cells” refers to chondrocytes, cartilage stem cells, or a combination thereof.

### Cartilage and Chondrocytes

Cartilage refers to cartilage tissue, which is composed of chondrocytes and intercellular substance. The matrix in the cartilage is in a gel-like state, endowing it with considerable toughness. Cartilage is a connective tissue that primarily serves a supportive function. There are no blood vessels and lymphatic vessels in cartilage. Nutrients infiltrate into the intercellular substance from the blood vessels in the perichondrium and then nourish bone cells.

Cartilage can be divided into 3 types according to the different intercellular substances, namely hyaline cartilage, elastic cartilage and fibrocartilage. The matrix of hyaline cartilage is composed of collagen fibers, fibrils and a surrounding amorphous matrix. During the embryonic period, it serves as a temporary scaffold, which is later replaced by bone. In adults, hyaline cartilage is mainly distributed in the trachea wall and bronchial wall, the sternal end of the ribs and the surface of the bone (articular cartilage). In addition to collagen fibers, there are elastic fibers in the matrix of elastic cartilage. This kind of cartilage has greater elasticity and is mainly distributed in the auricle, external auditory canal wall, eustachian tube, epiglottis, and larynx, etc. There are bundles of collagen fibers in the fibrocartilage matrix arranged in parallel or intersecting patterns, making the fibrocartilage matrix relatively tough.

Fibrocartilage is distributed in the intervertebral disc, glenoid cavity, articular disc, as well as in some tendons, and ligaments, etc., to enhance the flexibility of movement and protection, support and other functions.

In the preferred embodiment of the present invention, autologous chondrocytes (such as ear chondrocytes) obtained from a subject with cartilage defects are subcultured for 1-3 generations, and then subjected to in vitro concentrated incubation and chondrogenic induction culture to prepare injectable cartilage particles rich in extracellular matrix. The cartilage particles can be pre-molded in a customized mold or manually shaped after direct injection into the cartilage defect site, thus used for cartilage repair of cartilage defects.

### The Tissue Engineering Cartilage Particle and Graft of the Present Invention

The present invention provides an injectable tissue engineering cartilage particle comprising a cell population composed of chondrocytes and an extracellular matrix secreted by chondrocytes, wherein the extracellular matrix wraps around the cell population to form full and flattened spherical particles, and the average particle size of the cartilage particles is 500 μm-1 mm, wherein the density of chondrocytes in a single cartilage particle is at least 10⁴-10⁵ cells/cartilage particle.

The tissue engineering cartilage particles of the present invention are injectable tissue engineering cartilage particles, with an average particle size of 500 μm-1 mm per single cartilage particle. This size is smaller than the bore diameter of the syringe needle, allowing the tissue engineering cartilage particles to pass smoothly through the outlet of the syringe, thus they are referred to as injectable cartilage particles. The cartilage particles can not only be injected into a customized mold that matches the cartilage defect site for in vitro pre-shaping and transplanted to the subject's cartilage defect site, but also be directly injected into the cartilage defect site for artificial shaping. Compared to traditional tissue engineering cartilage grafts, the cartilage graft prepared with the cartilage particles of the present invention has a more uniform distribution of internal chondrocytes, does not introduce scaffold materials, and does not require mechanical cutting before use, thereby avoiding mechanical damage to chondrocytes before transplantation and the hidden danger of residual scaffold materials after transplantation. Therefore, it has a wider range of application prospects.

### Preparation Method of Cartilage Particle Graft of the Present Invention

The present invention also provides a method for preparing the cartilage particle graft according to the first aspect of the present invention. The preparation method of the tissue engineering cartilage graft of the present invention is simple and mainly comprises the following steps:
(1) Providing isolated chondrocytes for primary culture and subculture to obtain subcultured chondrocytes;
(2) Performing concentrated incubation culture on the subcultured chondrocytes obtained in step (1) in a concentrated incubation medium, to obtain chondrocyte micro-clusters;
(3) Culturing the chondrocyte micro-clusters for 3-5 days to obtain chondrocyte clusters;
(4) Performing in vitro chondrogenic induction culture of the chondrocyte clusters in a chondrogenic induction medium to obtain the tissue engineering cartilage particle.

In the above method, chondrocyte micro-clusters, where cartilage cells aggregate into clusters, are obtained by concentrated incubation of the subcultured chondrocytes in centrifuge tubes with conical, circular, or oval bottoms. The “chondrocyte micro-clusters” are further cultured to obtain chondrocyte clusters with an increased number of chondrocytes. As used herein, the term “concentrated incubation” refers to inoculating chondrocytes into a centrifuge tube containing a concentrated incubation medium, then placing the centrifuge tube vertically and statically in a 37° C., 5% CO₂ incubator, and continuing incubation after cell precipitation. The preferred incubation time is 6-12 hours. The obtained chondrocyte clusters can be subjected to chondrogenic induction culture in regular culture dishes to prepare the tissue engineering cartilage particles of the present invention. The duration of chondrogenic induction culture is 1-4 weeks, preferably 2-3 weeks, and most preferably 2 weeks. The particle size of the tissue engineering cartilage particles can be adjusted by controlling the time of in vitro chondrogenic induction culture.

### Therapeutic Application

The tissue engineering cartilage particles of the present invention can be used for repairing cartilage defects. In one embodiment of the present invention, the tissue engineering cartilage particles are injected into a customized mold that matches the cartilage defect site for pre-shaping, and then the shaped cartilage tissue graft is transplanted to the cartilage defect site. In another embodiment of the present invention, the tissue engineering cartilage particles are directly injected into the cartilage defect site and then artificially shaped.

In another preferred embodiment, the cartilage defect includes, but is not limited to a cartilage defect selected from the group consisting of: an articular cartilage defect, an auricular cartilage defect, a nasal cartilage defect, and a combination thereof.

In another preferred embodiment, the shape of the cartilage tissue graft is consistent or substantially consistent with the shape of cartilage injury in the human auricle, nasal dorsum, nasal wing, zygomatic arch, or superciliary arch.

In another preferred embodiment, the shape of the cartilage tissue graft includes, but is not limited to a shape selected from the group consisting of: tubular, rectangular, diamond-shaped, sheet-like, cylindrical, conical, spherical, and a combination thereof.

### The Main Advantages of the Present Invention Include:

(1) The preparation method of the tissue engineering cartilage particle graft of the present invention is simple, and there is no need for frequent medium changes during the preparation process. The consumption of culture factors is low, and the cost is relatively low.
(2) The tissue engineering cartilage particles of the present invention have a high yield. From only a few million (10⁶) cells inoculated into one 100 mm culture dish, 0.1-0.2 ml of injectable cartilage particles can be collected, and the culture time can be extended according to actual needs without affecting the injectability of the cartilage particles.
(3) The tissue engineering cartilage particle graft of the present invention is prepared without the introduction of scaffold materials, making it less prone to cause inflammatory reactions in the body and has no hidden danger of material residue.
(4) Compared to block cartilage constructed using injectable cartilage, the tissue engineering cartilage particle graft of the present invention does not require cutting before use, thus avoiding mechanical damage caused by sharp cutting.
(5) Compared to block cartilage constructed using injectable cartilage, the use of the tissue engineering cartilage particles of the present invention for transplantation can form a more homogeneous cartilage with no obvious cavities caused by nutrient deficiency within the tissue.

The present invention is further explained below in conjunction with specific example. It should be understood that these examples are only for illustrating the present invention and not intend to limit the scope of the present invention. The conditions of the experimental methods not specifically indicated in the following examples are usually in accordance with conventional conditions as described in Sambrook et al., Molecular Cloning: A Laboratory Manual (New York: Cold Spring Harbor Laboratory Press, 1989), or according to the conditions recommended by the manufacturer. Unless otherwise stated, percentages and parts are calculated by weight.

### Culture Medium

The medium used in the present invention are as follows:

Chondrocyte culture medium: DMEM high glucose (with a glucose content of 4.5 g/L) medium containing 10% serum (fetal bovine serum) and 1% triple antibiotic (containing 10000 units of penicillin, 10000 μg of streptomycin, and 25 μg of amphotericin B per milliliter).

Concentrated incubation medium: DMEM high glucose (with a glucose content of 4.5 g/L) medium containing 10% serum (fetal bovine serum) and 1% triple antibiotic (containing 10000 units of penicillin, 10000 μg of streptomycin, and 25 μg of amphotericin B per milliliter).

Chondrogenic induction medium: DMEM high glucose (with a glucose content of 4.5 g/L), 1% triple antibiotic (containing 10000 units of penicillin, 10000 μg of streptomycin, and 25 μg of amphotericin B per milliliter), 10 ng/ml TGF-β1 (R&D Systems Inc. Minneapolis, USA), 50 ng/ml IGF-I (R&D Systems Inc. Minneapolis, USA).

### Example 1: Preparation of Tissue Engineering Cartilage Particle Graft

The tissue engineering self-assembled cartilage particles of the present invention are prepared by the following method:
(1) The adherent and expanded chondrocytes (the passages 1-3 subcultured chondrocytes of primary chondrocytes) were digested with 0.25% trypsin and collected. The chondrocytes were then centrifuged at 1500 rpm, and the supernatant was removed. 20 mL of chondrocyte culture medium was added, and the cells were resuspended by pipetting.
(2) The resuspended cells were inoculated into a 50 mL sterile centrifuge tube at a density of 10×10⁶ cells/20 mL of a concentrated culture medium. The tube cap was gently closed but not tightly screwed to maintain sterile ventilation, then the tube was transferred to an incubator, and incubated upright at 37° C. with 5% CO₂ for 12 hours.
(3) The cells cultured in the 50 mL sterile centrifuge tube were evenly inoculated into a 10 cm×10 cm culture dish (with an inoculation density of 10×10⁶ cells/dish), and continued to be cultured in the concentrated culture medium for 3-5 days, with 1-2 changes of fresh medium during the culture period.
(4) After the formation of chondrocyte clusters (i.e., obtaining chondrocyte micro-clusters), the culture is switched to a chondrogenic induction medium for induction culture for 1 to 2 weeks.
(5) The culture obtained after chondrogenic induction culture was gently scraped off using a cell scraper and collected into a syringe. The excess culture medium was removed to obtain the tissue engineering cartilage particle graft.

The time required for in vitro chondrogenic induction culture was determined based on the desired particle diameter. Within 1-2 weeks, particles with a diameter of 0.5-1 mm could be formed. These particles could be easily scraped off and collected to obtain a scaffold-free cartilage particle graft with a certain degree of viscosity that could be used for injection.

TABLE 1
Cartilage particle size corresponding to chondrogenic
induction culture time

| Culture time for chondrogenesis | Cartilage particle size |
|---|---|
| 1 week | 0.25 mm |
| 2 weeks | 0.5 mm |

### Comparative Example 1

Example 1 was repeated, with the difference being that step (2) of culturing in a centrifuge tube was omitted, and the resuspended cells were directly cultured in a 10×10 cm culture dish for 12 hours+3-5 days.

Result: A tissue engineering cartilage particle graft could not be formed, instead, a thin cartilage slice was formed.

### Example 2: Animal Transplantation Experiment of Tissue Engineering Cartilage Particle Graft

The customized special shaped mold was placed in a culture dish after high-temperature sterilization for future use.

The cartilage particles collected and prepared in Example 1 were injected into a sterile mold, to fill the mold cavity (as shown in FIG. 3A). Chondrogenic induction medium was gently added to the mold, ensuring that the liquid level covered the top plane of the mold, and the mold was placed in an incubator for 2-4 weeks of continued incubation culture to obtain the tissue engineering cartilage particle graft (FIG. 3B).

Further harvesting or surgical implantation was performed under the skin of nude mice.

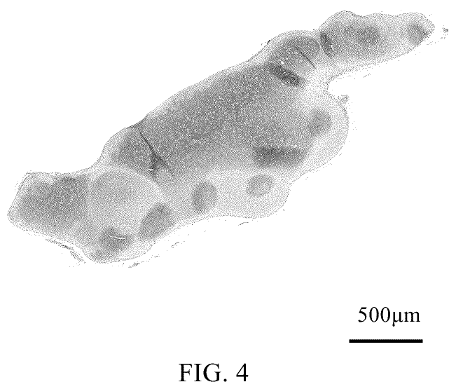

The results, as shown in FIGS. 3 and 4, indicated that after being implanted subcutaneously in animals for 4 weeks, the morphology of the tissue engineering cartilage particle graft could be basically maintained (FIG. 3C), and it was found that the graft regenerated cartilage tissue in the animal body (FIG. 3D). H-E staining also showed relatively homogeneous cartilage tissue regeneration (FIG. 4).

All references mentioned in the present application are incorporated by reference herein, as though individually incorporated by reference. In addition, it should be understood that after reading the above teaching content of the present invention, various changes or modifications may be made by those skilled in the art, and these equivalents also fall within the scope as defined by the appended claims of the present application.

1. A tissue engineering cartilage particle, which comprises a cell population composed of chondrocytes and an extracellular matrix secreted by chondrocytes, wherein the extracellular matrix wraps around the cell population to form full and flattened spherical particles, and the average particle size of the cartilage particle is 500 μm-1 mm, wherein the density of chondrocytes in a single cartilage particle is at least 10⁴-10⁵ cells per cartilage particle.

2. The cartilage particle of claim 1, wherein the cartilage particle is prepared by forming chondrocyte micro-clusters through concentrated incubation culture of chondrocytes, and then subjecting the chondrocyte micro-clusters to in vitro chondrogenic induction culture.

3. The cartilage particle of claim 2, wherein the “concentrated incubation” refers to inoculating a chondrocyte suspension into a centrifuge tube, subsequently placing the centrifuge tube vertically and statically in an incubator, and then continuing incubation after cell precipitation.

4. The cartilage particle of claim 3, wherein during the concentrated incubation, the chondrocyte suspension is inoculated at the following concentrations: 0.05×10⁶-20×10⁶ cells/mL of culture medium, preferably 0.1×10⁶-10×10⁶ cells/mL of culture medium, and more preferably 0.2×10⁶-5×10⁶ cells/mL of culture medium.

5. The cartilage particle of claim 3, wherein during the concentrated incubation, the culture system is 10-50 ml, preferably 15-30 ml, and more preferably about 20 mL of culture medium.

6. The cartilage particle of claim 3, wherein the bottom of the centrifuge tube is conical, circular, or oval shaped.

7. The cartilage particle of claim 2, wherein the culture medium used for concentrated incubation is a concentrated incubation medium, which comprises the following components: DMEM high glucose medium, 1% triple antibiotic (v/v), and 10% fetal bovine serum (v/v), calculated by the total volume of the culture medium.

8. The cartilage particle of claim 2, wherein the in vitro chondrogenic induction culture is performed using a chondrogenic induction medium, which comprises the following components: DMEM high glucose medium, 1% triple antibiotic (v/v), 10 ng/ml TGF-β, and 50 ng/ml IGF-I, calculated by the total volume of the culture medium.

9. The cartilage particle of claim 2, wherein the concentrated incubation time of chondrocytes is 3-24 hours, preferably 4-12 hours, and most preferably 6 hours.

10. The cartilage particle of claim 2, wherein the chondrogenic induction culture time is 1-4 weeks, preferably 2-3 weeks, and most preferably 2 weeks.

11. A method for preparing the tissue engineering cartilage particle of claim 1, which comprises the following steps:
(1) Providing isolated chondrocytes for primary culture and subculture to obtain subcultured chondrocytes;
(2) Performing concentrated incubation culture on the subcultured chondrocytes obtained in step (1) in a concentrated incubation medium, to obtain chondrocyte micro-clusters;
(3) Culturing the chondrocyte micro-clusters for 3-5 days to obtain chondrocyte clusters;
(4) Performing in vitro chondrogenic induction culture of the chondrocyte clusters in a chondrogenic induction medium to obtain the tissue engineering cartilage particle.

12. The method of claim 11, wherein the subcultured chondrocytes are chondrocytes from passages 1 to 3, preferably chondrocytes from passage 1.

13. A formulation or a transplantation composition, which comprises:
(a) the tissue engineering cartilage particle of claim 1; and
(b) a pharmaceutically acceptable carrier.

14. Use of the tissue engineering cartilage particle of claim 1 in the manufacture of a medical product for repairing a cartilage defect.

15. The use of claim 14, wherein the cartilage defect is selected from the group consisting of: an articular cartilage defect, an auricular cartilage defect, a nasal cartilage defect, and a combination thereof.
